(19) United States
(12) Patent Application Publication
(10) Pub. No.: US 2022/0059239 A1
(43) Pub. Date: Feb. 24, 2022

(54) IMAGE OR WAVEFORM ANALYSIS METHOD, SYSTEM AND NON-TRANSITORY COMPUTER-READABLE STORAGE MEDIUM

(71) Applicant: NantHealth, Inc., Culver City, CA (US)

(72) Inventor: Matheen M. Siddiqui, Culver City, CA (US)

(21) Appl. No.: 17/404,762

(22) Filed: Aug. 17, 2021

Related U.S. Application Data
(60) Provisional application No. 63/067,141, filed on Aug. 18, 2020.

(51) Int. Cl. G16H 50/70; G16H 10/60; G16H 30/40; G16H 50/20; G16H 70/60; G16H 50/30

(52) U.S. Cl. CPC G16H 50/70; G16H 10/60; G16H 50/30; G16H 50/20; G16H 70/60; G16H 30/40

(57) ABSTRACT

A method of interpreting images and/or waveforms determines differences between populations, input sources and/or test subjects. The method includes operations. The operations include receiving a first set of data from at least one of the input sources; encoding the first received data into a first lower dimensional representation; receiving a second set of data from the at least one of the input sources or from a second input source; encoding the second received data into a second lower dimensional representation; comparing the first low dimensional representation with the second low dimensional representation to generate a reconstruction; decoding the representation to reconstruct the data into a format similar to that of the received data; and transmitting a signal corresponding to the decoded representation. Related devices, apparatuses, systems, techniques, articles and non-transitory computer-readable storage medium are also described.

### CROSS REFERENCE TO RELATED APPLICATIONS

The present application claims the benefit of and priority to U.S. Provisional Application No. 63/067,141, filed on Aug. 18, 2020, the entire disclosure of which is hereby incorporated by reference.

### TECHNICAL FIELD

The present disclosure relates to a method, system and non-transitory computer-readable storage medium for analysis of images and/or waveforms. Specifically, the present disclosure relates to an architecture, devices, systems and related methods for analyzing, reproducing and detecting anomalies in images and/or waveforms including an electronic health record (EHR), an electrocardiogram (ECG), a speech waveform, a spectrogram, an electroencephalogram (EEG), and the like.

### BACKGROUND

Developed image and waveform analysis devices and methods include an attention mechanism for image captioning. The attention mechanism is a method of interpretable machine learning, which analyzes groups of data or components of input data and which may be used for internal steps of a classifier or decision making tool. The attention mechanism is described by Xu, K., Ba, J., Kiros, R., Cho, K., Courville, A., Salakhudinov, R., Zemel, R., & Bengio, Y. (2015, June), “Show, Attend and Tell: Neural Image Caption Generation with Visual Attention”, International Conference on Machine Learning (pp. 2048-2057).

FIGS. 14A to 14F depict three examples of input and output sets using an attention mechanism described by Xu et al. for image captioning in which an object of interest was successfully identified. With the attention mechanism for image captioning, patterns of pixels in images are correlated with identifiers (e.g., nouns). Specifically, an object in the image of FIG. 14A was highlighted as shown in FIG. 14B, and, using the attention mechanism for image captioning, the highlighted object was accurately identified as a “Frisbee”. Similarly, an object in the image of FIG. 14C was highlighted as shown in FIG. 14D, and, using the attention mechanism for image captioning, the highlighted object was accurately identified as a “dog”. Similarly, an object in the image of FIG. 14E was highlighted as shown in FIG. 14F, and, using the attention mechanism for image captioning, the highlighted object was accurately identified as a “stop sign”. However, there are limitations to the attention mechanism for image captioning.

FIGS. 15A to 15F depict three examples of input and output sets using the attention mechanism for image captioning in which an object of interest was not successfully identified. For example, an object in the image of FIG. 15A was highlighted as shown in FIG. 15B, and, using the attention mechanism for image captioning, the highlighted object was inaccurately identified as a “surfboard” (instead of a “windsail” or the like). Similarly, an object in the image of FIG. 15C was highlighted as shown in FIG. 15D, and, using the attention mechanism for image captioning, the highlighted object was inaccurately identified as a “pizza” (instead of “kebobs” or the like). Similarly, an object in the image of FIG. 15E was highlighted as shown in FIG. 15F, and, using the attention mechanism for image captioning, the highlighted object was inaccurately identified as a “cell phone” (instead of a “sandwich” or the like). In cases where the final result of a deep model is largely determined by groups or parts of input, attention as an interpretation mechanism may be effective for image captioning. However, a problem arises with use of the attention mechanism for image captioning, namely, accuracy may be limited. In some circumstances, the input must be discarded and replaced with a better image, i.e., a clearer image of the subject. Such replacement requires substantial subjective human verification and rework.

Also, the developed image and waveform analysis devices and methods including the attention mechanism have been used to analyze waveforms, such as ECGs and/or time series data. However, in time series data, like ECG, an entire signal is required with decisions being made on the basis of an overall shape. See, e.g., Mousavi, S., Afghah, F., & Acharya, U. R. (2020), “HAN-ECG: An Interpretable Atrial Fibrillation Detection Model Using Hierarchical Attention Networks”, arXiv preprint arXiv:2002.05262. FIG. 16A is an exemplary ECG input reported by Mousavi, et al., which, using the attention mechanism, was analyzed and highlighted resulting in the output shown in FIG. 16B. Portions of the ECG are highlighted and analyzed in FIG. 16B. The examples of FIGS. 16A and 16B were found to correspond with a subject without an atrial fibrillation (AF) arrhythmia. FIG. 17A is another exemplary ECG input reported by Mousavi, et al., which, using the attention mechanism, was analyzed and highlighted resulting in the output shown in FIG. 17B. Portions of the ECG are highlighted and analyzed in FIG. 17B. The examples of FIGS. 17A and 17B were found to correspond with a subject with an atrial fibrillation (AF) arrhythmia. That is, a problem arises with use of the attention mechanism for waveform analysis, namely, an entire input signal is required.

The present inventors developed improvements in devices and methods for analysis of images and/or waveforms that overcome at least the above-referenced problems with the devices and methods of the related art.

### SUMMARY

A method of interpreting images and/or waveforms to determine differences between populations, input sources and/or test subjects is provided.

A device may be provided. The device may have at least one processor and a memory storing at least one program for execution by the at least one processor. The at least one program may include instructions, which, when executed by the at least one processor cause the at least one processor to perform operations.

The operations may include receiving a first set of data from at least one of the input sources. The operations may include encoding the first received data into a first lower dimensional representation. The operations may include receiving a second set of data from the at least one of the input sources or from a second input source. The operations may include encoding the second received data into a second lower dimensional representation. The operations may include comparing the first low dimensional representation with the second low dimensional representation to generate a reconstruction. The operations may include decoding the representation to reconstruct the data into a format similar to that of the received data. The operations may include transmitting a signal corresponding to the decoded representation.

A system for interpreting images and/or waveforms to determine differences between populations, input sources and/or test subjects is provided. The system may include a device having at least one processor and a memory storing at least one program for execution by the at least one processor. The at least one program may include instructions, when, executed by the at least one processor cause the at least one processor to perform operations. The operations may include receiving a first set of data from at least one of the input sources. The operations may include encoding the first received data into a first lower dimensional representation. The operations may include receiving a second set of data from the at least one of the input sources or from a second input source. The operations may include encoding the second received data into a second lower dimensional representation. The operations may include comparing the first low dimensional representation with the second low dimensional representation to generate a reconstruction. The operations may include decoding the representation to reconstruct the data into a format similar to that of the received data. The operations may include transmitting a signal corresponding to the decoded representation.

A non-transitory computer-readable storage medium storing at least one program for interpreting images and/or waveforms to determine differences between populations, input sources and/or test subjects may be provided. The at least one program may be provided for execution by at least one processor and a memory storing the at least one program. The at least one program may include instructions, when, executed by the at least one processor cause the at least one processor to perform operations. The operations may include receiving a first set of data from at least one of the input sources. The operations may include encoding the first received data into a first lower dimensional representation. The operations may include receiving a second set of data from the at least one of the input sources or from a second input source. The operations may include encoding the second received data into a second lower dimensional representation. The operations may include comparing the first low dimensional representation with the second low dimensional representation to generate a reconstruction. The operations may include decoding the representation to reconstruct the data into a format similar to that of the received data. The operations may include transmitting a signal corresponding to the decoded representation.

Each of the method, system and non-transitory computer-readable storage medium may include one or more of the following features:

The first set of data and/or the second set of data may include one or more of an electronic health record (EHR), an electrocardiogram (ECG), a speech waveform, a spectrogram, and an electroencephalogram (EEG). The first set of data and/or the second set of data may include the ECG. Heart beats and fiducial markers may be identified in the decoded representation. An arrhythmia may be identified from the decoded representation. The first set of data and/or the second set of data may include the speech waveform. Differences relative to a standard pronunciation may be identified in the decoded representation. At least one anatomical structure may be associated with at least one segment of the decoded representation. At least one pathology may be associated with one or more segments of the decoded representation.

The first lower dimensional representation and/or the second lower dimensional representation may be encoded with one or more of a perturbation, a compactless loss, and a cross-entropy for classification.

The reconstruction may be generated with generative adversarial reconstruction (GAN).

The signal may be analyzed to highlight the differences between the populations, the input sources or the test subjects.

The signal may be analyzed with a decision exploration (DE) model to generate a decision.

The decision may include one or more of an admission decision, a readmission decision, a risk of mortality, and a diagnosis code. The diagnosis code may include an International Classification of Diseases (ICD) code.

The representation may be a blobby representation. The decoded representation may be a decoded blobby representation.

These and other capabilities of the disclosed subject matter will be more fully understood after a review of the following figures, detailed description, and claims.

### DESCRIPTION OF DRAWINGS

These and other features will be more readily understood from the following detailed description taken in conjunction with the accompanying drawings, in which:

FIG. 1 is a flow chart of a system for EHR analysis according to an exemplary embodiment;

FIG. 2A is a first exemplary sine wave signal according to an exemplary embodiment;

FIG. 2B is a first exemplary square wave signal according to an exemplary embodiment;

FIG. 2C is a schematic representation of a classifier according to an exemplary embodiment;

FIG. 3 is a schematic representation of a first encoder/decoder according to an exemplary embodiment;

FIG. 4 is a schematic representation of a second encoder/decoder according to an exemplary embodiment;

FIG. 5 is a schematic representation of a third encoder/decoder according to an exemplary embodiment;

FIG. 6A is a second exemplary sine wave signal according to an exemplary embodiment;

FIG. 6B is a third exemplary sine wave signal according to an exemplary embodiment;

FIG. 6C is a second exemplary square wave signal according to an exemplary embodiment;

FIG. 6D is a third exemplary square wave signal according to an exemplary embodiment;

FIG. 6E-1 is a first component of a first exemplary blobby representation of exemplary square wave signals according to an exemplary embodiment;

FIG. 6E-2 is a second component of the first exemplary blobby representation of the exemplary square wave signals according to an exemplary embodiment;

FIG. 7A is a fourth exemplary sine wave signal according to an exemplary embodiment;

FIG. 7B is a fifth exemplary sine wave signal according to an exemplary embodiment;

FIG. 7C is a sixth exemplary sine wave signal with a first local deformation according to an exemplary embodiment;

FIG. 7D is a seventh exemplary sine wave signal with a second local deformation according to an exemplary embodiment;

FIG. 7E is an eighth exemplary sine wave signal with a third local deformation according to an exemplary embodiment;

FIG. 7F is a ninth exemplary sine wave signal with a fourth local deformation according to an exemplary embodiment;

FIG. 7G is a tenth exemplary sine wave signal with a fifth local deformation according to an exemplary embodiment;

FIG. 7H is a second exemplary blobby representation of exemplary sine wave signals, some with local deformations according to an exemplary embodiment;

FIG. 7I-1 is a first component of the second exemplary blobby representation of the exemplary sine wave signals with the local deformations according to an exemplary embodiment;

FIG. 7I-2 is a second component of the second exemplary blobby representation of the exemplary sine wave signals with the local deformations according to an exemplary embodiment;

FIG. 8A is a third exemplary blobby representation according to an exemplary embodiment;

FIG. 8B is a component of the third exemplary blobby representation according to an exemplary embodiment;

FIG. 8C is a fourth exemplary blobby representation according to an exemplary embodiment;

FIG. 8D is a component of the fourth exemplary blobby representation according to an exemplary embodiment;

FIG. 9 is a schematic representation of a fourth encoder/decoder according to an exemplary embodiment;

FIG. 10A is a first exemplary step function signal according to an exemplary embodiment;

FIG. 10B is a second exemplary step function signal according to an exemplary embodiment;

FIG. 10C is a third exemplary step function signal according to an exemplary embodiment;

FIG. 10D is a fourth exemplary step function signal according to an exemplary embodiment;

FIG. 10E is a fifth exemplary step function signal according to an exemplary embodiment;

FIG. 10F is a fifth exemplary blobby representation according to an exemplary embodiment;

FIG. 10G is the fifth exemplary blobby representation with an asymptote removed according to an exemplary embodiment;

FIG. 11 is a flow chart of a system for generative adversarial reconstruction (GAN) analysis according to an exemplary embodiment;

FIG. 12 is a flow chart of a method for waveform analysis according to an exemplary embodiment;

FIG. 13 is a schematic diagram of a computer device or system including at least one processor and a memory storing at least one program for execution by the at least one processor according to an exemplary embodiment;

FIG. 14A is a first exemplary input image for a prior art image analysis method;

FIG. 14B is a first exemplary output image for the prior art image analysis method;

FIG. 14C is a second exemplary input image for the prior art image analysis method;

FIG. 14D is a second exemplary output image for the prior art image analysis method;

FIG. 14E is a third exemplary input image for the prior art image analysis method;

FIG. 14F is a third exemplary output image for the prior art image analysis method;

FIG. 15A is a fourth exemplary input image for the prior art image analysis method;

FIG. 15B is a fourth exemplary output image for the prior art image analysis method;

FIG. 15C is a fifth exemplary input image for the prior art image analysis method;

FIG. 15D is a fifth exemplary output image for the prior art image analysis method;

FIG. 15E is a sixth exemplary input image for the prior art image analysis method;

FIG. 15F is a sixth exemplary output image for the prior art image analysis method;

FIG. 16A is a first exemplary input image for a prior art waveform analysis method;

FIG. 16B is a first exemplary output image for the prior art waveform analysis method;

FIG. 17A is a second exemplary input image for a prior art waveform analysis method; and

FIG. 17B is a second exemplary output image for the prior art waveform analysis method.

It is noted that the drawings are not necessarily to scale. The drawings are intended to depict only typical aspects of the subject matter disclosed herein, and therefore should not be considered as limiting the scope of the disclosure. Those skilled in the art will understand that the structures, systems, devices, and methods specifically described herein and illustrated in the accompanying drawings are non-limiting exemplary embodiments and that the scope of the present invention is defined solely by the claims.

### DETAILED DESCRIPTION

Reconstructions of data, including blobby reconstructions and wiggle plots, are described and provided, which overcome problems with the developed methods of analysis of images and/or waveforms. The present reconstructions do not have the accuracy problems of the attention mechanism, nor do the present reconstructions require an entirety or a substantial entirely of a given input signal. Also, the present reconstructions permit visualizations that inform a viewer of how a shape of a signal informs a given outcome.

The present devices and methods may be applied to generate interpretable classification of heart beats as well as fiducial detection. A wiggle plot may be used to show how heart beats are classified as having arrhythmias or not. The present devices and methods may model and visualize global as well as local changes of a waveform to aid in clinical decisions.

From a data analysis/visualization point of view, the present devices and methods may learn and/or visualize the differences between populations, learn and/or visualize the difference between sensors (sensing devices), learn and/or visualize differences between a single person over time, and the like.

The present devices and methods may be used with speech therapy, by visualizing the difference between a patient's current pronunciation of a word and a collection of standard pronunciations. The present devices and methods may be used to train a classifier for a particular word or phrase. A wiggle plot (e.g., for a spectrogram) may be used to allow the patient to develop visual insight into a difference between how the patient speaks versus a standard pronunciation. For instance, such visualizations would be useful to deaf patients. The present devices and methods may help patients target efforts toward a particular goal. A speech therapist may correlate certain outputs of the present devices and methods to parts and positions of the human anatomy (e.g., mouth, throat, tongue, etc.) to permit a patient to focus on and/or correct a current difference to achieve a desired result.

Targeted visualization may be used with EEG signals, both to visualize differences in terms of pathology, data analysis, and to help a patient produce a certain signal.

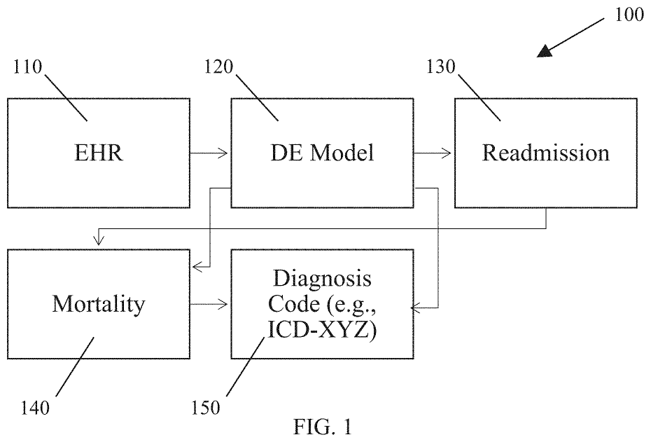

FIG. 1 is a flow chart of a system 100 for electronic health record (EHR) analysis according to an exemplary embodiment. The system 100 may include input of an EHR 110. The system 100 may include analysis of the EHR using a decision exploration model 120. The system 100 may include making a readmission decision 130 based on the decision exploration model 120. The system 100 may include determining a mortality rate 140 based on the decision exploration model 120, and/or the readmission decision 130. The system 100 may include a determination of a diagnosis code (e.g., International Statistical Classification of Diseases and Related Health Problems (ICD) XYZ format) 150 based on the EHR 110, and/or the decision exploration model 120, and/or the readmission decision 130, and/or the mortality rate 140. The system 100 may be trained from data after diagnosis coding. The system 100 may be used in a clinical setting to offer an interpretable mechanism for predicting outcomes. The system 100 may be used as a decision support tool. For example, the system 100 may determine what part of the EHR 110 correlates with a particular outcome. The system 100 may capture trends in decision making and outcomes.

The system 100 may be configured to collect all decisions that are being made in the EHR 110 at a backend of any given process. The system 100 may be configured to prompt a doctor or health care professional to update the EHR 110 when a patient comes to a health care facility. The system 100 may be configured to display possibilities, an identify parts of the EHR 110, which are more likely than average to result in a particular outcome. The system 100 allows the user to understand what is happening and/or identify particular events to watch out for by highlighting or displaying a given word, phrase, sentence or image.

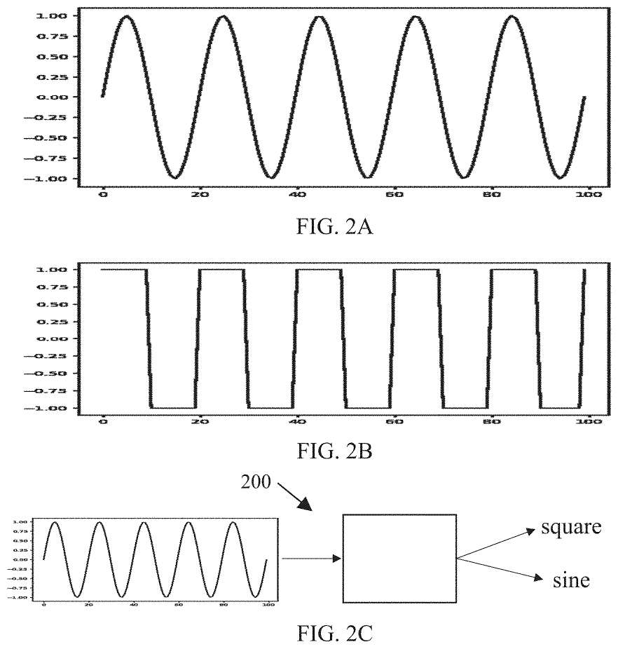

A system 300 for generating a wiggle plot is provided. The system 300 may be configured to determine how to minimally change an input to flip a decision while maintaining an appearance that is similar to the original input. For example, consider a simplistic example of a square wave versus a sine wave. FIG. 2A is a first exemplary sine wave signal according to an exemplary embodiment. FIG. 2B is a first exemplary square wave signal according to an exemplary embodiment. FIG. 2C is a schematic representation of a classifier 200 according to an exemplary embodiment. The classifier 200 may receive as input a waveform (in this case, a sine wave), and the classifier 200 may output an identification of whether the waveform is a sine wave or a square wave. The system 300 may include the classifier 200. The system 300 may be configured to perform a formulation step. The formulation step may include an embedding step and a learning differences step.

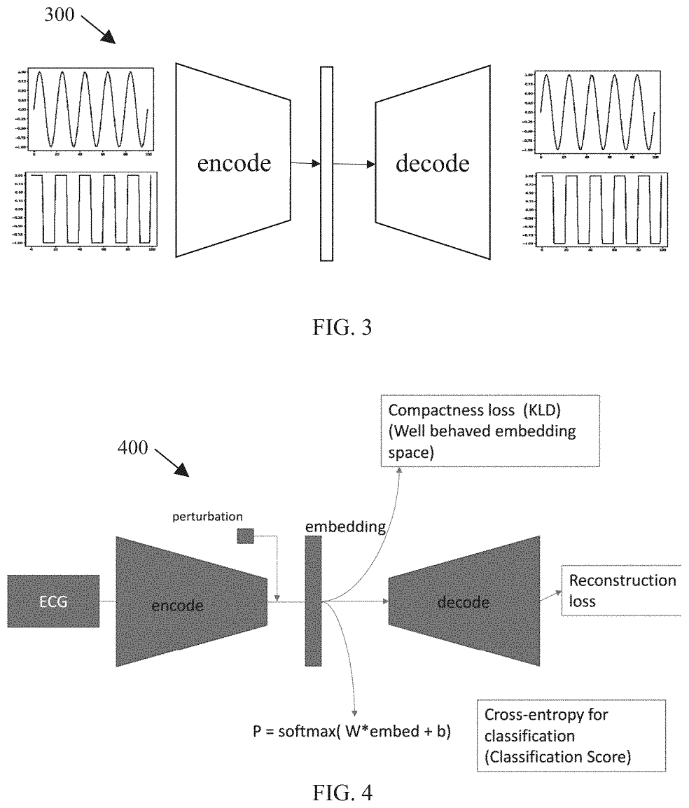

The embedding step may be represented by FIG. 3, which is a schematic representation of the system 300, which may include a first encoder/decoder according to an exemplary embodiment. The system 300 may include an auto-encoding step. The system 300 may incorporate machine learning. The system 300 may be configured to receive one or more input signals, apply a transform to reduce the one or more input signals to a low-dimensional representation of the one or more input signals, and then apply another transform to reconstruct the one or more input signals. The encoder transform and the decoder transform may be learnt and/or solved to reconstruct the one or more input signals. The low-dimensional representation may represent a signal space.

An interpretable embedding step may be represented by FIG. 4, which is a schematic representation of a system 400, which may include a second encoder/decoder according to an exemplary embodiment. The system 400 may include an embedding that not only preserves a signal space, but also includes additional properties. These additional properties may be enforced in a training loss. The training loss may include a reconstruction loss and/or a classification loss. With the reconstruction loss, the embedding may represent a signal, since the signal may be reconstructed from the embedding. With the classification loss, the embedding may be separable by a linear classifier, so the embedding may contain normal information and/or abnormal information. The embedding space may be compact and/or smooth. Perturbations and/or compactless loss (e.g., Kullback-Leibler divergence (KLD)) may be used in variational auto-encoding. Compactless loss or KLD are examples of well-behaved embedding spaces. The embedding step may include a word embedding model, such as Softmax, i.e., P=softmax (W*embed+b). Cross-entropy for classification may be used to generate a classification score.

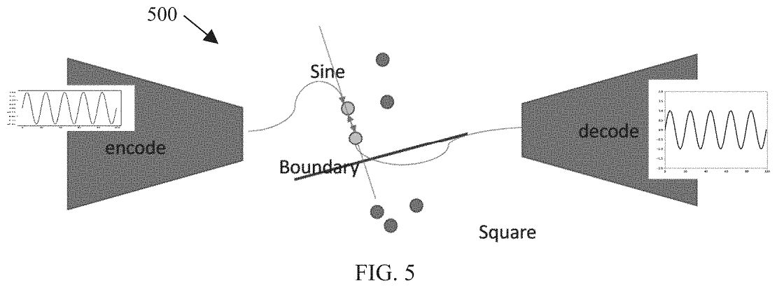

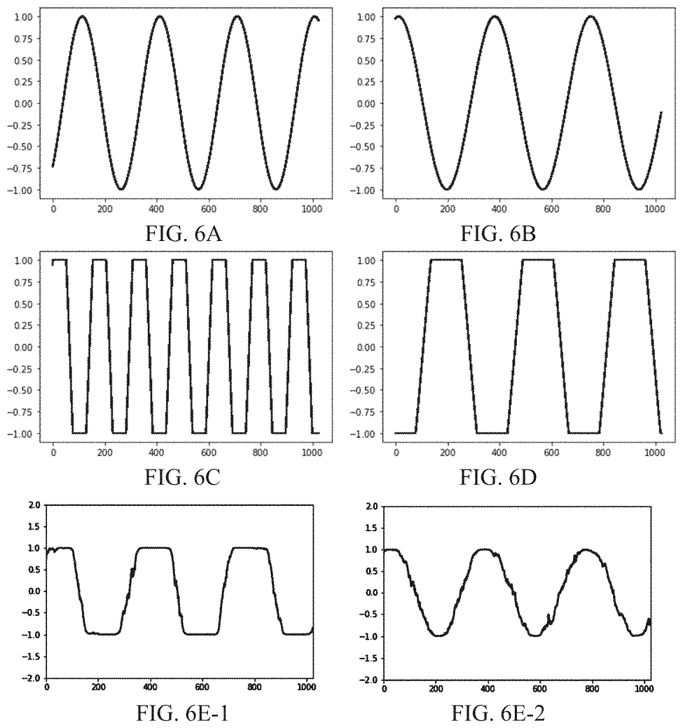

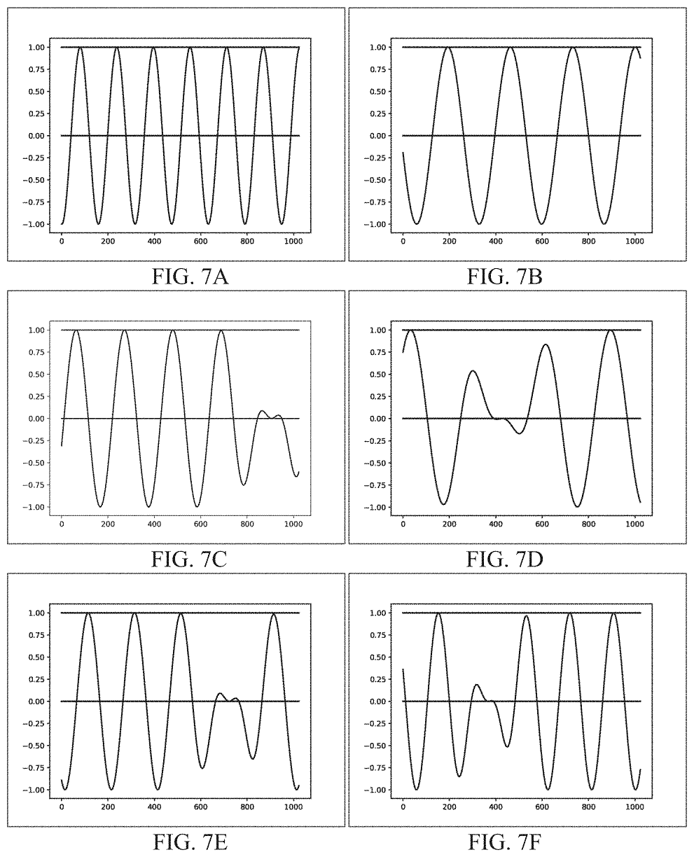

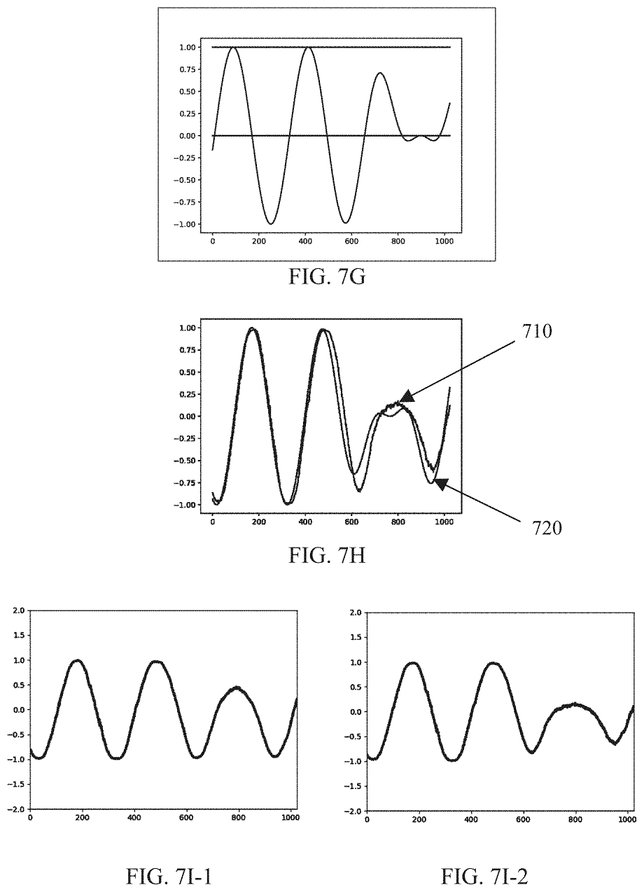

Another interpretable embedding step may be represented by FIG. 5, which is a schematic representation of a system 500, which may include a third encoder/decoder according to an exemplary embodiment. The embedding space of the system 500 may be separable by a linear classifier due to a classification loss. A sample may be moved in a given direction toward a decision boundary, to make the sample look like another class of sample. The direction of movement may correspond with a wiggle direction. A signal may be recovered from the embedding space. The recovered signal may show how a global difference and/or a local difference affects a shape of a signal. Specifically, the output of the decoder may show how a global difference and/or a local difference affects a shape of a signal. FIGS. 6 and 7 provide examples of classes with a global difference and/or a local difference. Samples from two classes and a wiggle plot are provided. The wiggle plot may animate a sample as it appears through a decoder when the sample is moved toward the decision boundary.

An example of a global shape between classes is provided. FIG. 6A is a second exemplary sine wave signal according to an exemplary embodiment. FIG. 6B is a third exemplary sine wave signal according to an exemplary embodiment. FIG. 6C is a second exemplary square wave signal according to an exemplary embodiment. FIG. 6D is a third exemplary square wave signal according to an exemplary embodiment.

FIGS. 6E-1 and 6E2 are first and second components, respectively, of a first exemplary blobby representation of exemplary square wave signals according to an exemplary embodiment. The images of FIGS. 6E-1 and 6E2 were generated by taking an example square wave, then transforming the example square wave into an embedded space, i.e., an original embedding. Then, from the original embedding, a direction toward a decision boundary is found. Then, the embedding is changed and moved closer to the decision boundary, i.e., a perturbed embedding. Both the original embedding and the perturbed embedding are reconstructed through the decoder. An animation, e.g., an animated GIF, may be displayed, which may, for example, alternate between the images of FIGS. 6E-1 and 6E2 to show differences between the original embedding and the perturbed embedding. The animated GIF may include two or more images. The animated GIF may effectively trick an eye of a human observer into seeing a relatively smooth and/or pulsing transition back and forth between the at least two images.

The direction to the decision boundary can be found in a number of ways. For example, a difference between an average location of a sine waveform embedding and an average location of a square/chopped off embedding may be determined and used to inform the determination of the direction of the decision boundary.

An example of localized deformation is provided. FIG. 7A is a fourth exemplary sine wave signal without a localized deformation according to an exemplary embodiment. FIG. 7B is a fifth exemplary sine wave signal without a localized deformation according to an exemplary embodiment. FIG. 7C is a sixth exemplary sine wave signal with a first local deformation (centered at about 900 units along the x-axis) according to an exemplary embodiment. FIG. 7D is a seventh exemplary sine wave signal with a second local deformation (centered at about 400 units along the x-axis) according to an exemplary embodiment. FIG. 7E is an eighth exemplary sine wave signal with a third local deformation (centered at about 700 units along the x-axis) according to an exemplary embodiment. FIG. 7F is a ninth exemplary sine wave signal with a fourth local deformation (centered at about 350 units along the x-axis) according to an exemplary embodiment. FIG. 7G is a tenth exemplary sine wave signal with a fifth local deformation (centered at about 900 units along the x-axis) according to an exemplary embodiment.

FIG. 7H is a second exemplary blobby representation of exemplary sine wave signals, some with local deformations according to an exemplary embodiment. FIG. 7H includes an overlay of an original wave form 710 and a reconstructed wave form 720.

The images of FIGS. 7I-1 and 7E-2 may be generated by a method similar to that described above with respect to FIGS. 6E-1 and 6E-2, respectively. With FIGS. 7I-1 and 7I-2, in addition to global shape changes as shown, for example, in FIGS. 6E-1 and 6E-2, the blobby representation captures local changes. That is, FIGS. 7I-1 and 7I-2, a difference between a group that includes normal sine waves (e.g., FIGS. 7A and 7B) and a group with localized attenuation (e.g., FIGS. 7C-7G, inclusive) can be visualized.

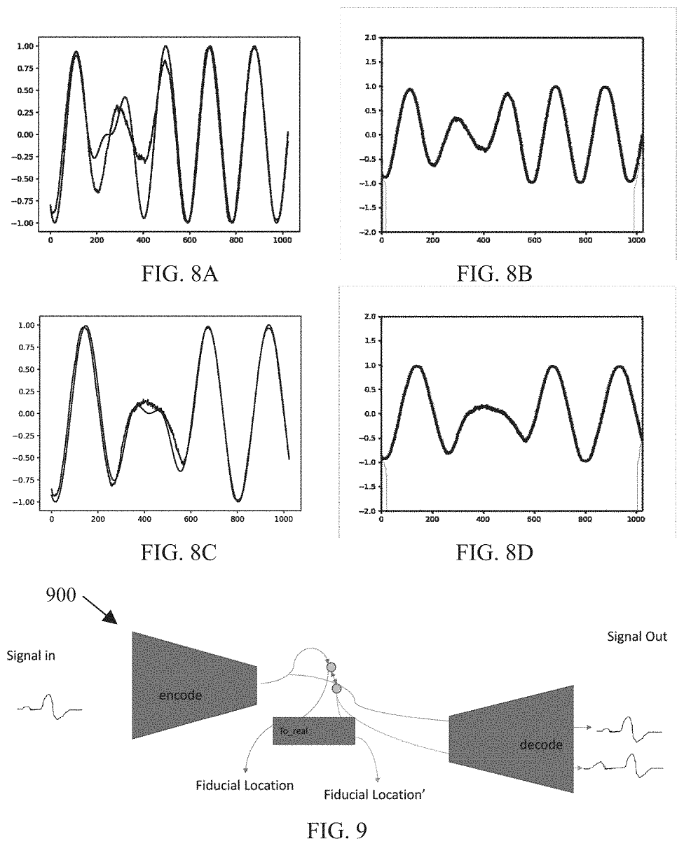

FIG. 8A is a third exemplary blobby representation according to an exemplary embodiment. FIG. 8B is a component of the second exemplary wiggle representation according to an exemplary embodiment. FIG. 8A is similar to FIG. 7H.

FIG. 8C is a fourth exemplary blobby representation according to an exemplary embodiment. FIG. 8D is a component of the fourth exemplary blobby representation according to an exemplary embodiment. FIG. 8C is similar to FIG. 7H.

An interpretation of a real-valued output may be represented by FIG. 9, which is a schematic representation of a system 900, which may include a fourth encoder/decoder according to an exemplary embodiment. The system 900 may include a step of learning a mapping from the embedding space to a target value using a “To_real” function. The system 900 may be configured to generate a wiggle plot or a blobby representation by finding embedding that moves to another target value, with minimal change to the reconstructed signal according to the following: Signal2 embedding=argmin_embedding[to_real(embedding)+lambda||Decode(embedding)−signal||]. The output of the To_real function may include one or more fiducial locations of the input signal, and an anticipated fiducial location (i.e., fiducial location’) of the output signal.

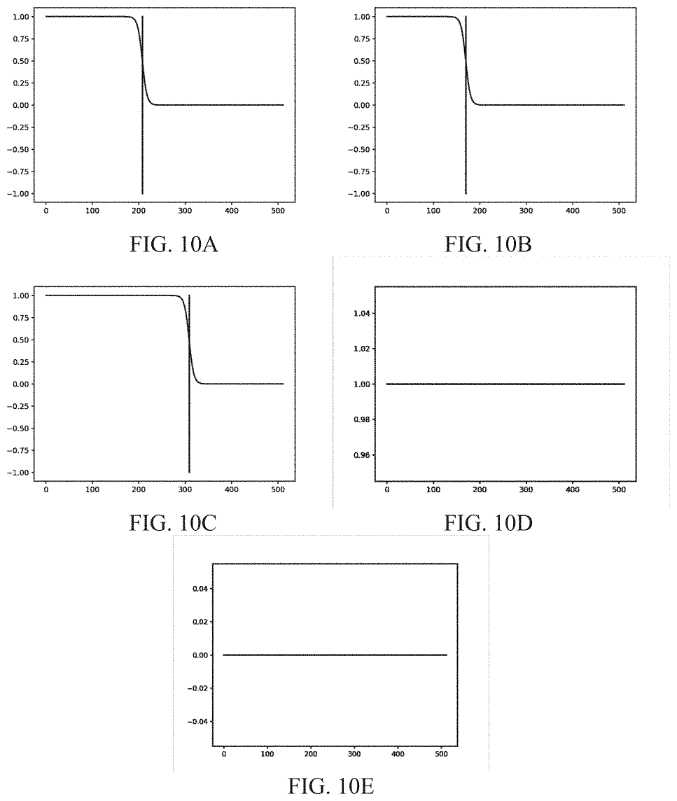

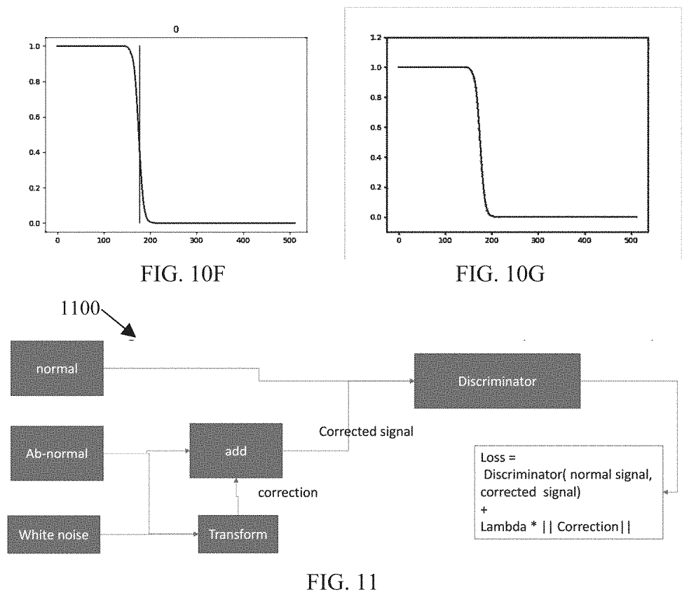

Using a step function as an example, FIG. 10A is a first exemplary step function signal (positive sample having a step edge) according to an exemplary embodiment. FIG. 10B is a second exemplary step function signal (positive sample having a step edge) according to an exemplary embodiment. FIG. 10C is a third exemplary step function signal (positive sample having a step edge) according to an exemplary embodiment. FIG. 10D is a fourth exemplary step function signal (negative sample, which is flat) according to an exemplary embodiment. FIG. 10E is a fifth exemplary step function signal (negative sample, which is flat) according to an exemplary embodiment. FIG. 10F is a fourth exemplary wiggle representation of the first, second, third, fourth, and fifth exemplary step function signals according to an exemplary embodiment. FIG. 10G is the fourth exemplary wiggle representation with an asymptote removed according to an exemplary embodiment.

FIG. 11 is a flow chart of a system 1100 for generative adversarial reconstruction (GAN) analysis according to an exemplary embodiment. The previous examples included directions in an embedding space that both separated a class and reconstructed a signal. With the GAN analysis, point by point, a sample is transformed such that the sample looks like the sample was drawn from an opposing set. The system 1100 may use GAN analysis to learn a difference between signals. One or more abnormalities in a signal may be corrected to generate a normal signal for comparison. The GAN analysis searches for a smallest signal that can be added to an abnormal signal to make the abnormal signal into a normal signal. Loss may be expressed with the following formula: Loss=Discriminator(normal signal, corrected signal)+Lambda*||Correction||.

The system 1100 may be configured to repeatedly solve two problems. First, the system 1100 may be configured to determine a discriminator that can separate samples from “normal” and “corrected signals”. The discriminator may be a deep learning model. With the deep learning model, optimization over samples of data may amplify the differences. The system 1100 may be configured to determine a best Transform that minimizes the differences between samples of “corrected signals” and “normal”. The system 1100 may be configured to minimize a magnitude of the correction to have a minimal correction. The system 1100 may be configured with a Transform that produces a signal, and the signal may be analyzed, point by point, to add to the “abnormal signal” such that the change has minimal magnitude, and such that the resulting signal looks like “normal”.

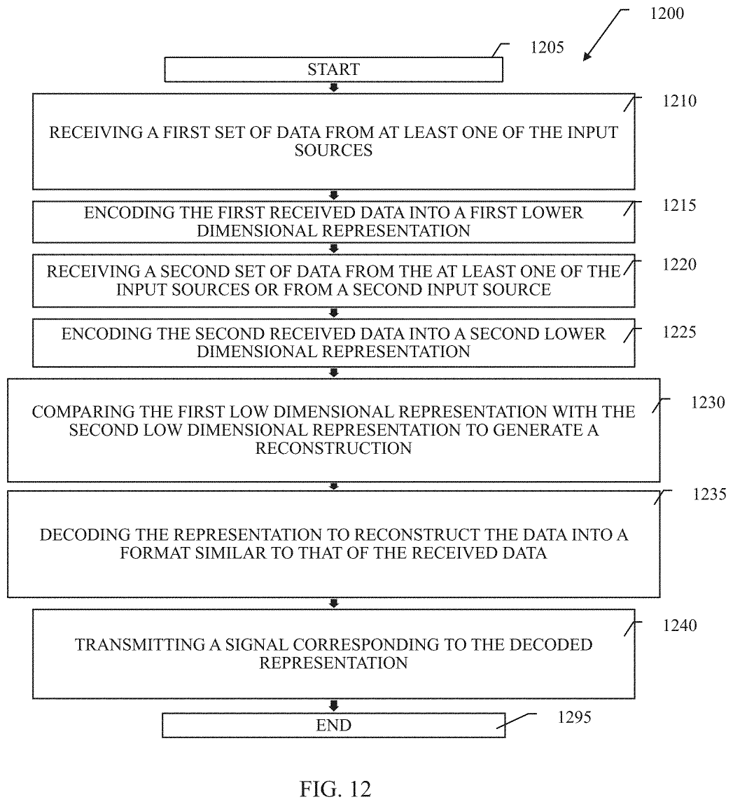

FIG. 12 is a flow chart of a method 1200 for image and/or waveform analysis according to an exemplary embodiment. The method 1200 may include a start 1205 and an end 1295. The method 1200 may include receiving a first set of data from at least one of the input sources (1210). The method 1200 may include encoding the first received data into a first lower dimensional representation (1215). The method 1200 may include receiving a second set of data from the at least one of the input sources or from a second input source (1220). The method 1200 may include encoding the second received data into a second lower dimensional representation (1225). The method 1200 may include comparing the first low dimensional representation with the second low dimensional representation to generate a reconstruction (1230). The method 1200 may include decoding the representation to reconstruct the data into a format similar to that of the received data (1235). The method 1200 may include transmitting a signal corresponding to the decoded representation (1240).

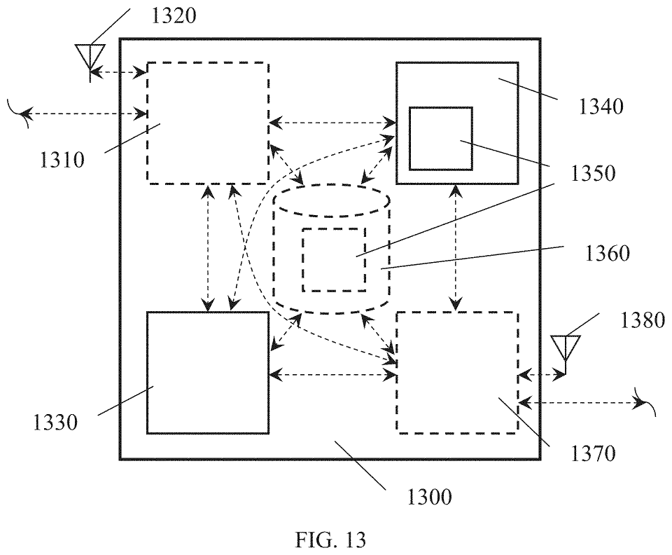

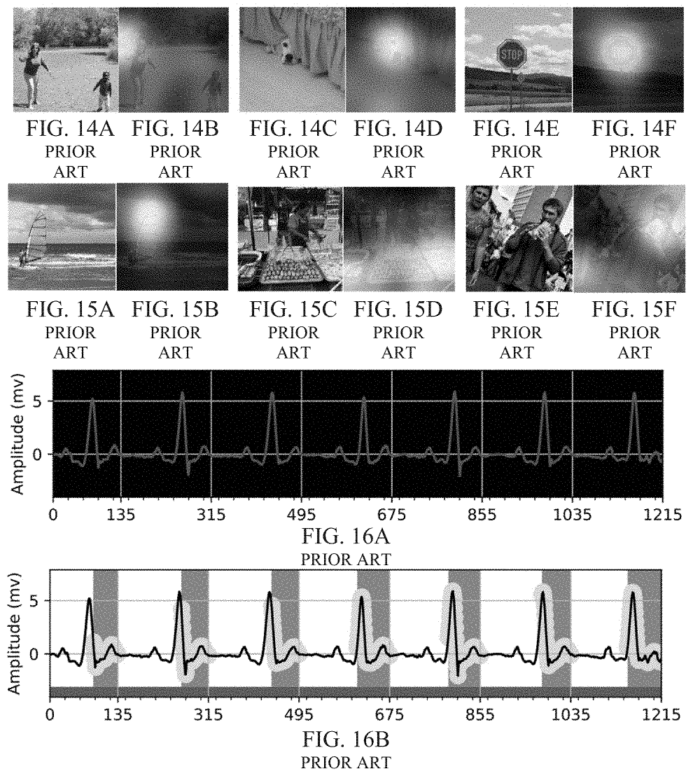

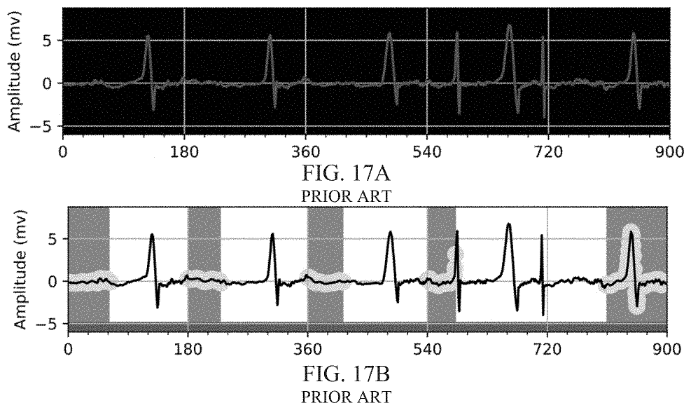

FIG. 13 is a schematic diagram of a computer device or system including at least one processor and a memory storing at least one program for execution by the at least one processor according to an exemplary embodiment. Specifically, FIG. 13 depicts a computer device or system 1300 comprising at least one processor 1330 and a memory 1340 storing at least one program 1350 for execution by the at least one processor 1330. In some embodiments, the device or computer system 1300 can further comprise a non-transitory computer-readable storage medium 1360 storing the at least one program 1350 for execution by the at least one processor 1330 of the device or computer system 1300. In some embodiments, the device or computer system 1300 can further comprise at least one input device 1310, which can be configured to send or receive information to or from any one of: an external device (not shown), the at least one processor 1330, the memory 1340, the non-transitory computer-readable storage medium 1360, and at least one output device 1370. The at least one input device 1310 can be configured to wirelessly send or receive information to or from the external device via a means for wireless communication, such as an antenna 1320, a transceiver (not shown) or the like. In some embodiments, the device or computer system 1300 can further comprise at least one output device 1370, which can be configured to send or receive information to or from any one from the group consisting of: an external device (not shown), the at least one input device 1310, the at least one processor 1330, the memory 1340, and the non-transitory computer-readable storage medium 1360. The at least one output device 1370 can be configured to wirelessly send or receive information to or from the external device via a means for wireless communication, such as an antenna 1380, a transceiver (not shown) or the like.

Each of the above identified modules or programs corresponds to a set of instructions for performing a function described above. These modules and programs (i.e., sets of instructions) need not be implemented as separate software programs, procedures or modules, and thus various subsets of these modules may be combined or otherwise re-arranged in various embodiments. In some embodiments, memory may store a subset of the modules and data structures identified above. Furthermore, memory may store additional modules and data structures not described above.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described herein can include electrical circuit(s) that can include components and circuitry elements of suitable value in order to implement the embodiments of the subject innovation(s). Furthermore, it can be appreciated that many of the various components can be implemented on at least one integrated circuit (IC) chip. For example, in one embodiment, a set of components can be implemented in a single IC chip. In other embodiments, at least one of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that at least one component may be combined into a single component providing aggregate functionality or divided into several separate sub-components, and any at least one middle layer, such as a management layer, may be provided to communicatively couple to such sub-components in order to provide integrated functionality. Any components described herein may also interact with at least one other component not specifically described herein but known by those of skill in the art.

In addition, while a particular feature of the subject innovation may have been disclosed with respect to only one of several implementations, such feature may be combined with at least one other feature of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with at least one specific functionality. For example, a component may be, but is not limited to being, a process running on a processor (e.g., digital signal processor), a processor, an object, an executable, a thread of execution, a program, and/or a computer. By way of illustration, both an application running on a controller and the controller can be a component. At least one component may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used herein differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by at least one local or remote computing device, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has at least one of its characteristics set or changed in such a manner as to encode information in at least one signal. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described herein. Furthermore, not all illustrated acts may be required to implement the methodologies in accordance with the disclosed subject matter. In addition, those skilled in the art will understand and appreciate that the methodologies could alternatively be represented as a series of interrelated states via a state diagram or events. Additionally, it should be appreciated that the methodologies disclosed in this specification are capable of being stored on an article of manufacture to facilitate transporting and transferring such methodologies to computing devices. The term article of manufacture, as used herein, is intended to encompass a computer program accessible from any computer-readable device or storage media.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Although at least one exemplary embodiment is described as using a plurality of units to perform the exemplary process, it is understood that the exemplary processes may also be performed by one or plurality of modules.

The use of the terms “first”, “second”, “third” and so on, herein, are provided to identify various structures, dimensions or operations, without describing any order, and the structures, dimensions or operations may be executed in a different order from the stated order unless a specific order is definitely specified in the context.

Approximating language, as used herein throughout the specification and claims, may be applied to modify any quantitative representation that could permissibly vary without resulting in a change in the basic function to which it is related. Accordingly, a value modified by a term or terms, such as “about” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein may be embodied in systems, apparatus, methods, and/or articles depending on the desired configuration. The embodiments set forth in the foregoing description do not represent all embodiments consistent with the subject matter described herein. Instead, they are merely some examples consistent with aspects related to the described subject matter. Although a few variations have been described in detail above, other modifications or additions are possible. In particular, further features and/or variations may be provided in addition to those set forth herein. For example, the embodiments described above may be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other embodiments may be within the scope of the following claims.

What is claimed is:

1. A method of interpreting images and/or waveforms to determine differences between populations, input sources and/or test subjects, wherein a device is provided, the device having at least one processor and a memory storing at least one program for execution by the at least one processor, the at least one program including instructions, which, when executed by the at least one processor cause the at least one processor to perform operations comprising:
receiving a first set of data from at least one of the input sources;
encoding the first received data into a first lower dimensional representation;
receiving a second set of data from the at least one of the input sources or from a second input source;
encoding the second received data into a second lower dimensional representation;
comparing the first low dimensional representation with the second low dimensional representation to generate a reconstruction;
decoding the representation to reconstruct the data into a format similar to that of the received data; and
transmitting a signal corresponding to the decoded representation.

2. The method of claim 1, wherein the first set of data and/or the second set of data comprises one or more of an electronic health record (EHR), an electrocardiogram (ECG), a speech waveform, a spectrogram, and an electroencephalogram (EEG).

3. The method of claim 2, wherein the first set of data and/or the second set of data comprises the ECG, and wherein heart beats and fiducial markers are identified in the decoded representation.

4. The method of claim 2, wherein the first set of data and/or the second set of data comprises the ECG, and wherein an arrhythmia is identified from the decoded representation.

5. The method of claim 2, wherein the first set of data and/or the second set of data comprises the speech waveform, and wherein differences relative to a standard pronunciation are identified in the decoded representation.

6. The method of claim 2, wherein the first set of data and/or the second set of data comprises the speech waveform, and wherein at least one anatomical structure is associated with at least one segment of the decoded representation.

7. The method of claim 2, wherein the first set of data and/or the second set of data comprises the EEG, and wherein at least one pathology is associated with one or more segments of the decoded representation.

8. The method of claim 1, wherein the first lower dimensional representation and/or the second lower dimensional representation is encoded with one or more of a perturbation, a compactless loss, and a cross-entropy for classification.

9. The method of claim 1, wherein the reconstruction is generated with generative adversarial reconstruction (GAN).

10. The method of claim 1, wherein the signal is analyzed to highlight the differences between the populations, the input sources or the test subjects.

11. The method of claim 1, wherein the signal is analyzed with a decision exploration (DE) model to generate a decision.

12. The method of claim 11, wherein the decision includes one or more of an admission decision, a readmission decision, a risk of mortality, and a diagnosis code.

13. The method of claim 12, wherein the diagnosis code comprises an International Classification of Diseases (ICD) code.

14. The method of claim 1, wherein the representation is a blobby representation, and the decoded representation is a decoded blobby representation.

15. A system for interpreting images and/or waveforms to determine differences between populations, input sources and/or test subjects, the system comprising:
a device having at least one processor and a memory storing at least one program for execution by the at least one processor, the at least one program including instructions, when, executed by the at least one processor cause the at least one processor to perform operations comprising:
receiving a first set of data from at least one of the input sources;
encoding the first received data into a first lower dimensional representation;
receiving a second set of data from the at least one of the input sources or from a second input source;
encoding the second received data into a second lower dimensional representation;
comparing the first low dimensional representation with the second low dimensional representation to generate a reconstruction;
decoding the representation to reconstruct the data into a format similar to that of the received data; and
transmitting a signal corresponding to the decoded representation.

16. The system of claim 15, wherein the first set of data and/or the second set of data comprises one or more of an electronic health record (EHR), an electrocardiogram (ECG), a speech waveform, a spectrogram, and an electroencephalogram (EEG).

17. The system of claim 16, wherein the first set of data and/or the second set of data comprises the ECG, and wherein heart beats and fiducial markers are identified in the decoded representation.

18. The system of claim 16, wherein the first set of data and/or the second set of data comprises the ECG, and wherein an arrhythmia is identified from the decoded representation.

19. The system of claim 16, wherein the first set of data and/or the second set of data comprises the speech waveform, and wherein differences relative to a standard pronunciation are identified in the decoded representation.

20. The system of claim 16, wherein the first set of data and/or the second set of data comprises the speech waveform, and wherein at least one anatomical structure is associated with at least one segment of the decoded representation.

21. The system of claim 16, wherein the first set of data and/or the second set of data comprises the EEG, and wherein at least one pathology is associated with one or more segments of the decoded representation.

22. The system of claim 15, wherein the first lower dimensional representation and/or the second lower dimensional representation is encoded with one or more of a perturbation, a compactless loss, and a cross-entropy for classification.

23. The system of claim 15, wherein the reconstruction is generated with generative adversarial reconstruction (GAN).

24. The system of claim 15, wherein the signal is analyzed to highlight the differences between the populations, the input sources or the test subjects.

25. The system of claim 15, wherein the signal is analyzed with a decision exploration (DE) model to generate a decision.

26. The system of claim 25, wherein the decision includes one or more of an admission decision, a readmission decision, a risk of mortality, and a diagnosis code.

27. The system of claim 26, wherein the diagnosis code comprises an International Classification of Diseases (ICD) code.

28. The system of claim 15, wherein the representation is a blobby representation, and the decoded representation is a decoded blobby representation.

29. A non-transitory computer-readable storage medium storing at least one program for interpreting images and/or waveforms to determine differences between populations, input sources and/or test subjects, the at least one program for execution by at least one processor and a memory storing the at least one program, the at least one program including instructions, when, executed by the at least one processor cause the at least one processor to perform operations comprising:
receiving a first set of data from at least one of the input sources;
encoding the first received data into a first lower dimensional representation;
receiving a second set of data from the at least one of the input sources or from a second input source;
encoding the second received data into a second lower dimensional representation;
comparing the first low dimensional representation with the second low dimensional representation to generate a reconstruction;
decoding the representation to reconstruct the data into a format similar to that of the received data; and
transmitting a signal corresponding to the decoded representation.

30. The non-transitory computer-readable storage medium of claim 29, wherein the first set of data and/or the second set of data comprises one or more of an electronic health record (EHR), an electrocardiogram (ECG), a speech waveform, a spectrogram, and an electroencephalogram (EEG).

31. The non-transitory computer-readable storage medium of claim 30, wherein the first set of data and/or the second set of data comprises the ECG, and wherein heart beats and fiducial markers are identified in the decoded representation.

32. The non-transitory computer-readable storage medium of claim 30, wherein the first set of data and/or the second set of data comprises the ECG, and wherein an arrhythmia is identified from the decoded representation.

33. The non-transitory computer-readable storage medium of claim 30, wherein the first set of data and/or the second set of data comprises the speech waveform, and wherein differences relative to a standard pronunciation are identified in the decoded representation.

34. The non-transitory computer-readable storage medium of claim 30, wherein the first set of data and/or the second set of data comprises the speech waveform, and wherein at least one anatomical structure is associated with at least one segment of the decoded representation.

35. The non-transitory computer-readable storage medium of claim 30, wherein the first set of data and/or the second set of data comprises the EEG, and wherein at least one pathology is associated with one or more segments of the decoded representation.

36. The non-transitory computer-readable storage medium of claim 29, wherein the first lower dimensional representation and/or the second lower dimensional representation is encoded with one or more of a perturbation, a compactless loss, and a cross-entropy for classification.

37. The non-transitory computer-readable storage medium of claim 29, wherein the reconstruction is generated with generative adversarial reconstruction (GAN).

38. The non-transitory computer-readable storage medium of claim 29, wherein the signal is analyzed to highlight the differences between the populations, the input sources or the test subjects.

39. The non-transitory computer-readable storage medium of claim 29, wherein the signal is analyzed with a decision exploration (DE) model to generate a decision.

40. The non-transitory computer-readable storage medium of claim 39, wherein the decision includes one or more of an admission decision, a readmission decision, a risk of mortality, and a diagnosis code.

41. The non-transitory computer-readable storage medium of claim 40, wherein the diagnosis code comprises an International Classification of Diseases (ICD) code.

42. The non-transitory computer-readable storage medium of claim 29, wherein the representation is a blobby representation, and the decoded representation is a decoded blobby representation.
